(19) United States
(12) Patent Application Publication
(10) Pub. No.: US 2015/0190773 A1
(43) Pub. Date: Jul. 9, 2015

(54) PROCESS FOR PREPARING ETHYLENE AND/OR PROPYLENE

(71) Applicants: Leslie Andrew Chewter, Amsterdam (NL); Ye-Mon Chen, Sugar Land, TX (US); Timothy Michael Nisbet, Amsterdam (NL); Sivakumari Sadasivan Vijayakumari, Gonzales, LA (US); Jeroen Van Westrenen, Amsterdam (NL)

(72) Inventors: Leslie Andrew Chewter, Amsterdam (NL); Ye-Mon Chen, Sugar Land, TX (US); Timothy Michael Nisbet, Amsterdam (NL); Sivakumari Sadasivan Vijayakumari, Gonzales, LA (US); Jeroen Van Westrenen, Amsterdam (NL)

(21) Appl. No.: 14/412,693

(22) PCT Filed: Jul. 1, 2013
(86) PCT No.: PCT/EP2013/063837

§ 371 Date: Jan. 5, 2015

Related U.S. Application Data
(60) Provisional application No. 61/667,628, filed on Jul. 3, 2012.

(51) Int. Cl. B01J 8/00; B01J 38/12; B01J 8/08; B01J 29/90; C07C 1/20; C07C 4/06

(52) U.S. Cl. CPC B01J 8/0055; C07C 1/20; C07C 4/06; B01J 8/08; B01J 29/90; B01J 38/12; C07C 2529/40; B01J 2208/00761; B01J 2208/00752

(57) ABSTRACT

A process for preparing ethylene and/or propylene, wherein oxygenates and olefins are converted to ethylene and/or propylene over a zeolite-comprising catalyst, in two reaction steps. The catalyst is circulated in the reaction system.

### FIELD OF THE INVENTION

This invention relates to a process for preparing ethylene and/or propylene, a reaction system suitable therefore and use of the reaction system.

### BACKGROUND TO THE INVENTION

Conventionally, ethylene and propylene are produced via steam cracking of paraffinic feedstocks including ethane, propane, naphtha and hydrowax. An alternative route to ethylene and propylene is an oxygenate-to-olefin (OTO) process. Interest in OTO processes for producing ethylene and propylene is growing in view of the increasing availability of natural gas. Methane in the natural gas can be converted, for instance, to methanol or dimethylether (DME), both of which are suitable feedstocks for an OTO process.

In an OTO process, an oxygenate such as methanol is provided to a reaction zone comprising a suitable conversion catalyst and converted to ethylene and propylene. In addition to the desired ethylene and propylene, a substantial part of the methanol is converted to higher hydrocarbons including C4+ olefins.

These C4+ olefins may be recycled and provided together with the oxygenate to the OTO reaction zone. Such a process is for instance described in U.S. Pat. No. 6,441,261, wherein it is mentioned that C4+ hydrocarbon mixtures that are obtained from separation and recycle of the reaction product are co-fed to the reactor together with the oxygenate.

In WO2009/156433, an alternative is proposed to recycling the C4+ fraction in the reaction product to the reaction zone to be co-fed together with the oxygenate. In order to increase the ethylene and propylene yield of the process, WO2009/156433 proposes to further crack the C4+ olefins in a dedicated olefin cracking zone to produce further ethylene and propylene. In WO2009/156433, a process is described, wherein an oxygenate feedstock is converted in an OTO zone (XTO zone) to an ethylene and propylene product. Higher olefins, i.e. C4+ olefins, produced in the OTO zone are directed to an olefin cracking zone (OC zone). In the olefin cracking zone, part of the higher olefins is converted to additional ethylene and propylene to increase the overall yield of the process to ethylene and propylene. While the conversion of the oxygenate feedstock is an exothermic process, the conversion of the higher olefins in the OC zone is an endothermic process.

In WO2009/156433, the catalyst is circulated from the OC zone to the XTO zone. Catalyst exiting the XTO zone is passed to a regenerator. The catalyst deactivates during use due to the formation of carbon deposits on the catalyst. These are removed in the regenerator, where these carbon deposits are combusted with oxygen, typically in the form of air, at elevated temperatures.

This regeneration step is exothermic and resultantly the temperature of the catalyst increases during regeneration. The hot catalyst is subsequently provided to the OC zone, thereby providing the heat required for the endothermic conversion of the higher olefins in the OC zone.

Although the process of WO2009/156433 integrates the oxygenate conversion process and the olefin cracking process by using the same catalyst, which is cycled from the olefin cracking process to the oxygenate conversion process and, via the regenerator back to the olefin cracking zone, the process is sensitive to upsets in one or more of the reaction zones. In addition, the design of the process of WO2009/156433 does not allow variation of the flow of catalyst provided to either the olefin cracking process or the oxygenate conversion process without inevitably also changing the flow of catalyst provided to the other process.

Where the catalyst activity is virtually restored by the regeneration of the catalyst, continuous regeneration at high temperatures results in an irreversible deactivation of the catalyst on a longer time scale. At the high temperature conditions that prevail in the catalyst regenerator the zeolites in the catalyst may typically undergo a hydrothermal degradation, whereby the zeolite structure is damaged.

WO2009/156433 suggests that part of the catalyst exiting the XTO zone may bypass the regenerator and be provided together with hot catalyst, which was regenerated, to the OC zone. However, as the regenerator provides the heat required for the endothermic conversion of the higher olefins in the OC zone, the catalyst which was regenerated, has to provide more heat and must be heated to even higher temperatures. Moreover, such a catalyst cycle, whereby both catalyst exiting the XTO zone and hot catalyst from the regenerator are provided to the OC zone, makes the process highly sensitive to deliberate or non-deliberate changes to the conditions in the XTO zone.

The selectivity of the oxygenate conversion in the XTO zone is not negatively influenced, and may even be improved, by coke on catalyst; however, the selectivity of the olefin cracking in the OC zone is sensitive to coke on catalyst. Ideally, any catalyst provided to an olefin cracking process such as in the OC zone of WO2009/156433 is hot, clean catalyst. Clean herein means that the catalyst comprises low levels of coke, but does not necessarily mean the catalyst is free of coke. However, in the process of WO2009/156433 it is suggested to send catalyst from the XTO zone, i.e. catalyst with a high coke on catalyst, directly to the OC zone, which may negatively influence the olefin cracking selectivity.

A further disadvantage of the process of WO2009/156433 is that the process requires that both the XTO zone and the OC zone have a separate gas/solid section to separate the products from the catalyst.

There is a need in the art for an improved integrated process for producing ethylene and propylene from an oxygenate feed, wherein the oxygenate conversion and olefin cracking are integrated by cycling the catalyst between the oxygenate conversion and the olefin cracking, with intermediate regeneration of the catalyst and wherein the interdependency of the olefin cracking process and the oxygenate conversion process is reduced.

### SUMMARY OF THE INVENTION

It has now been found that an improved integrated process for producing ethylene and propylene from an oxygenate feed, wherein the oxygenate conversion and olefin cracking are integrated by cycling the catalyst between the oxygenate conversion and the olefin cracking, with intermediate regeneration of the catalyst, may be obtained by recycling part of the catalyst exiting the OTO reactor back to the OTO reactor and providing the remainder to the regenerator, while catalyst that is regenerated is provided to both the OTO as well as the OCP reactor.

Accordingly, the present invention provides a process for preparing ethylene and/or propylene, wherein oxygenates and olefins are converted to ethylene and/or propylene over a zeolite-comprising catalyst, comprising the steps of:

a) reacting in a first reactor an oxygenate feed over the zeolite-comprising catalyst at a temperature in the range of from 350 to 700° C. and retrieving from the first reactor a first reactor effluent stream comprising gaseous products, including ethylene and/or propylene, and zeolite-comprising catalyst;
b) reacting in a second reactor an olefin feed over the zeolite-comprising catalyst at a temperature in the range of from 500 to 700° C. and retrieving from the second reactor a second reactor effluent stream comprising gaseous products, including ethylene and/or propylene, and zeolite-comprising catalyst;
c) providing the first and second reactor effluent stream to one or more gas/solid separators to retrieve zeolite-comprising catalyst from the first and second reactor effluent;
d) providing part of the zeolite-comprising catalyst retrieved in step (c) to the first reactor;
e) regenerating another part of the zeolite-comprising catalyst retrieved in step (c) by contacting the zeolite-comprising catalyst with oxygen at elevated temperatures to provide a hot regenerated zeolite-comprising catalyst; and
f) providing part of the hot regenerated zeolite-comprising catalyst to the first reactor and another part of the hot regenerated zeolite-comprising catalyst to the second reactor.

Reference herein to an oxygenate feedstock is to a feedstock comprising oxygenates.

Reference herein to an olefin feedstock is to a feedstock comprising olefins, in particular to a feedstock comprising C4+ olefins, i.e. olefins comprising 4 or more carbon atoms.

Reference herein to hot regenerated zeolite-comprising catalyst in step (e) and (f) is to zeolite-comprising catalyst having a higher temperature than the zeolite-comprising catalyst provided to the regenerator in step (e).

The conversion of the oxygenate feedstock over a zeolite-comprising catalyst to at least ethylene and/or propylene in step (a) is also referred to as an oxygenate to olefin (OTO) process. Such OTO processes are well known in the art.

The conversion of the olefin feedstock over a zeolite-comprising catalyst to at least ethylene and/or propylene in step (a) is also referred to as an olefin cracking process (OCP). Such OCP processes are well known in the art.

The process according to the invention reduces the sensitivity of the process to changes in one or more of its sub-processes, i.e. the OCP and/or OTO processes, while allowing the same catalyst inventory to be used for both the OCP and OTO process.

The current invention does not require all or part of the catalyst to be heated to temperatures higher than necessary to achieve the desired regeneration of the catalyst, even when part of the catalyst bypasses the regenerator. Endothermic conversion processes rely on the external provision of heat. In prior art processes, this heat is provided via the catalyst. The amount of heat provided to the endothermic conversion of an olefin feed is a function of the temperature and mass flow of catalyst coming from two different processes, i.e. an OTO process and a catalyst regeneration process. The process according to the invention, wherein only hot regenerated catalyst from the regenerator is provided to the OCP reactor is less sensitive to changes in the operating condition in the OTO process.

The process according to the invention allows for the use of one gas/solid separation unit, i.e. a gas/solid separator, for both the effluent of the OTO reactor and the OCP reactor, reducing complexity and capital cost.

The process according to the present invention has the advantage that the total effluent exiting OTO reactor and the OCP reactor may be cooled to prevent undesired formation of by-products. Where, in the prior-art processes, part of the catalyst exiting OTO reactor is provided to the OCP reactor, such cooling would be undesirable as a hot catalyst is required to provide heat for the endothermic OCP reaction.

Prior art processes, in particular a process such as disclosed in WO2009/156433, are cascaded, i.e., regenerated catalyst goes to OCP, OCP spent catalyst goes to OTO, OTO spent catalyst goes to regenerator, with the exception of an optional partial by-pass from OTO to OCP in particular process layouts. In the process according to the present invention, the catalyst circulation is parallel, i.e. regenerated catalyst goes to both OCP and OTO spent catalyst goes to regenerator, with the exception of the partial by-pass of mixed spent catalyst to OTO. Reference herein to OTO spent catalyst is to catalyst that exits the OTO reactor. Reference herein to OCP spent catalyst is to catalyst that exits the OCP reactor. Reference herein to mixed spent catalyst is to catalyst that exits the gas/solid separator.

Due to the nature of the parallel catalyst circulation in the process according to the present invention, an independent control of two catalyst streams to OTO and OCP is achieved. This allows for the individual optimization of the catalyst to hydrocarbon ratio for both the OTO and the olefin cracking processes, which is particularly relevant in case riser reactors are used. This catalyst to hydrocarbon ratio is typically referred to as the cat/oil ratio. For the purpose of calculating the cat/oil ratio, the term hydrocarbon is to be interpreted as hydrocarbon including oxygenate. Consequently, in the process according to the invention, it is possible to increase the cat/oil in OTO process, while at the same time decrease cat/oil to the olefin cracking process. Due to the cascade nature of the catalyst circulation in prior art processes, it is not possible to independently optimize the cat/oil ratios for both the OTO and olefin cracking process. By using the process according to the present invention it is possible to increase cat/oil in OTO process, without necessarily having to increase the cat/oil to OCP process at the same time.

In the process according to the invention, rather than sending catalyst directly from the OTO reactor, i.e. catalyst that is high on coke, the catalyst provided to the OCP reactor is hot and clean as it comes directly from the regenerator. Clean herein means that the catalyst comprises low levels of coke, but does not necessarily mean the catalyst is free of coke.

In another aspect the invention provides a reaction system suitable for preparing ethylene and propylene, comprising

a) a first reactor;
b) a second reactor;
c) a regenerator; and
d) a gas/solid separator;
wherein the reaction system further comprises:
means for providing a first reactor effluent stream from the first reactor to the gas/solid separator;
means for providing a second reactor effluent stream from the second reactor to the gas/solid separator;
means for providing catalyst from the gas/solid separator to the regenerator;
means for providing regenerated catalyst from the regenerator to the first reactor;
means for providing regenerated catalyst from the regenerator to the second reactor; and
means for providing catalyst from the gas/solid separator to the first reactor.

The invention further provides the use of the reaction system according to the invention in a process according to the invention.

### BRIEF DESCRIPTION OF THE DRAWING

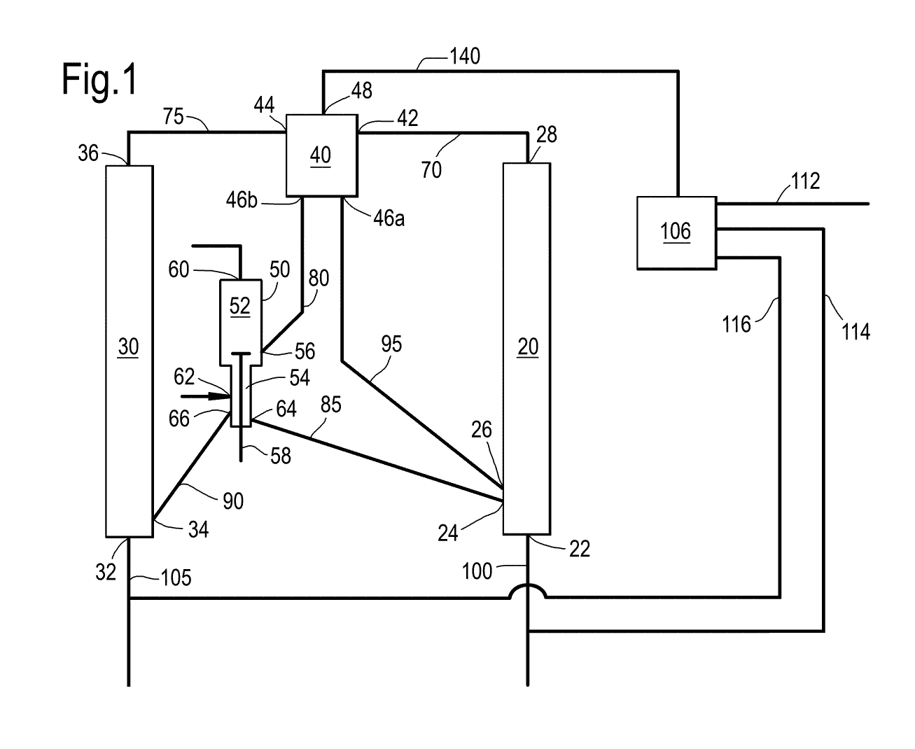

In FIG. 1 an embodiment of a system for preparing ethylene and/or propylene according to the invention is shown.

### DETAILED DESCRIPTION OF THE INVENTION

Ethylene and/or propylene can be produced from oxygenates such as methanol and dimethylether (DME) through an oxygenate-to-olefins (OTO) process. Such processes are well known in the art and are also referred to as methanol-to-olefins or methanol-to-propylene processes. In an OTO process, typically the oxygenate is contacted with a zeolite-comprising catalyst at elevated temperatures. In contact with the zeolite-comprising catalyst, the oxygenate is converted into ethylene and/or propylene. Besides ethylene and propylene, substantial amounts of C4+ olefins are produced. To increase the total yield of ethylene and propylene, these C4+ olefins may be converted to obtain further ethylene and propylene. One way of converting the C4+ olefins to ethylene and propylene is through cracking the C4+ olefins by contacting the C4+ olefins at elevated temperature with a zeolite-comprising catalyst. This process is generally referred to as an olefin cracking process or OCP.

Both the OTO process as well as the olefin cracking process may use the same zeolite-comprising catalyst. This zeolite-comprising catalyst may be cycled between the OTO process step and the OCP process step, while intermediately at least part of the catalyst is regenerated.

Due to different enthalpic properties of the OTO process and the OCP process, i.e. the OTO process is exothermic while the OCP process is endothermic, it is necessary to maintain a good heat balance between OTO, OCP and catalyst regeneration processes. As heat is transported from one process step to another via the catalyst, the circulation of the catalyst is essential to maintaining the correct heat balance. In addition, distribution of the catalyst over the OTO, OCP and catalyst regeneration processes is essential to the flexibility of the process.

In the process according to the present invention, ethylene and/or propylene are prepared by converting oxygenates and olefins over a zeolite-comprising catalyst. In a first reactor, an oxygenate feed is converted over the zeolite-comprising catalyst at a temperature in the range of from 350 to 700° C. The first reactor is also referred to as the OTO reactor and the process that takes place in the first reactor is referred to as an OTO process. In contact with the zeolite-comprising catalyst, at least part of the oxygenates in the oxygenate feed are converted to a gaseous product, which includes at least ethylene and/or propylene and preferably both. In addition to ethylene and/or propylene, the gaseous product may comprise higher olefins, i.e. C4+ olefins, and paraffins. The gaseous product is retrieved from the first reactor as part of a first reactor effluent stream. This effluent additionally comprises zeolite-comprising catalyst.

In a second reactor, an olefin feed is converted over the zeolite-comprising catalyst at a temperature in the range of from 500 to 700° C. The second reactor is also referred to as the OCP reactor and the process that takes place in the first reactor is referred to as an olefin cracking process. In contact with the zeolite-comprising catalyst, at least part of the olefins in the olefin feed is converted to a gaseous product, which includes at least ethylene and/or propylene and preferably both. In addition to ethylene and/or propylene, the gaseous product may comprise higher olefins, i.e. C4+ olefins, and paraffins. The gaseous product is retrieved from the second reactor as part of a second reactor effluent stream. This effluent additionally comprises zeolite-comprising catalyst.

Preferably, the first and/or second reactors are riser reactors. More preferably, the first and second reactors are riser reactors. The advantage of the use of a riser reactor is that it allows for very accurate control of the contact time of the several feeds with the catalyst, as riser reactors exhibit a flow of catalyst and reactants through the reactor that approaches plug flow.

The first and second reactor effluent, individually, comprise zeolite-comprising catalyst and a gaseous product, comprising ethylene and propylene. The reactor effluent comprises advantageously at least 50 mol %, in particular at least 50 wt %, ethylene and propylene, based on total hydrocarbon content in the reactor effluent.

The first and second reactor effluent streams are subsequently provided to one or more gas/solid separators to retrieve zeolite-comprising catalyst from the first and second reactor effluent.

The first and second reactor effluent streams may separately be sent to two separate gas/solid separators each set to receive either the first or the second effluent streams. This may for instance be beneficial, where the first and second reactors are fluidized bed type reactors. Such fluidized bed type reactors typically have internal gas/solid separators built into the reactor housing. Preferably, however, the first and second reactor effluent streams are provided, at least partially, to the same gas/solid separator. This is particularly advantageous where the first and/or second reactors are riser reactors. Such riser reactors typically rely on external gas/solid separators to separate the catalyst from the gaseous product and can therefore be combined with a joint gas/solid separator. Additionally, this may reduce the CAPEX of the process.

The gas/solid separator may be any separator suitable for separating gases from solids. Preferably, the gas/solid separator comprises one or more centrifugal or cyclone, preferably cyclone, units, optionally combined with a stripper section.

Preferably, the reactor effluent is cooled in the gas/solid separator. It is particularly advantageous to cool the reactor effluent in order to terminate the conversion process, being either the OTO or olefin cracking process. By termination of the conversion processes post reaction and the resulting formation of by-products outside the reactors is prevented as much as possible. Preferably, the cooling in the gas/solid separator is achieved by a water quench. When the reactors used in process of WO2009/156433 are riser reactors, the process of WO2009/156433 has the disadvantage that the effluent of the OC zone, i.e. a mixture of catalyst and the products, exits the OC zone still having an elevated temperature similar to the temperature conditions inside the OC zone. The high temperature of this mixture may lead to undesired reaction involving the products and may result in the formation of by-products and coke. As result of the formation of by-products and coke, selectivity of the process is decreased. In conventional processes using riser reactors, the effluent of a riser reactor is typically cooled upon exiting the riser reactor to quench the reaction and to prevent any undesired side-reactions. However, in the process of WO2009/156433 the effluent from OC zone cannot be cooled significantly. As described herein above, the catalyst exiting the OC zone is provided to the XTO zone, without intermediate heating of the catalyst in the regenerator. Cooling of the OC zone effluent, including the catalyst, would cause the catalyst temperature of the catalyst to drop below the catalyst temperature required for the XTO reaction. Consequently, in the process of WO2009/156433 the effluent of the OC-zone must first be separated to retrieve the catalyst, thereby increasing the risk of by-product formation.

Following the separation of zeolite-comprising catalyst from the gaseous product, part of the zeolite-comprising catalyst retrieved in step (c) is provided to the first reactor. This gas/solid zeolite-comprising catalyst still comprises carbon deposits as it has not been regenerated, which is beneficial to the selectivity of the OTO process. It is known in the art that the presence of coke deposits on the zeolite-comprising catalyst may positively influence the OTO process. In addition, preferably, the zeolite-comprising catalyst has a lower temperature compared to the zeolite-comprising catalyst which was retrieved from the first reactor as part of the first reactor effluent stream. This may in part have been achieved by mixing the catalyst in the first reactor effluent stream with the catalyst in the second reactor effluent stream retrieved from the endothermic OCP process. Preferably, the zeolite-comprising catalyst provided from the gas/solid separator to the first reactor has a lower temperature compared to the zeolite-comprising catalyst which was retrieved from the first reactor as part of the first reactor effluent stream because it was actively cooled in the gas/solid separator. Optionally, the zeolite-comprising catalyst provided from the gas/solid separator to the first reactor has a lower temperature compared to the zeolite-comprising catalyst which was retrieved from the first reactor as part of the first reactor effluent stream because either the first or second reactor effluent stream was cooled before entering the gas/solid separator or the zeolite-comprising catalyst provided from the gas/solid separator was cooled before entering the first reactor.

Preferably, the temperature of the zeolite-comprising catalyst retrieved in step (c) is in the range of from 10 to 100° C., more preferably of from 50 to 95° C. below the temperature of the zeolite-comprising catalyst which was retrieved from the first reactor as part of the first effluent. This zeolite-comprising catalyst having a lower temperature may be used to absorb part of heat produced by the exothermic OTO reaction.

Another part, and preferably the remainder, of the zeolite-comprising catalyst retrieved in step (c) is provided and subsequently regenerated in a catalyst regeneration process. Preferably, in the range of from 0.33 to 0.67 wt % of the zeolite-comprising catalyst retrieved in step (c) is provided and subsequently regenerated in a catalyst regeneration process As mentioned herein above, during the OTO and olefin cracking processes carbon is deposited on the catalysts, which results in a, albeit reversible, deactivation of the zeolite-comprising catalyst. In the catalyst regeneration process the zeolite-comprising catalyst is contacted with oxygen at elevated temperatures, typically in the range of from 500 to 700° C., preferably of from 550 to 650° C. During the regeneration process the carbon deposits are at least partially removed by combustion with the oxygen. Preferably, the oxygen is provided as air or oxygen-enriched air. This combustion is an exothermic process leading to a temperature increase of the zeolite-comprising catalyst. The catalyst retrieved from the catalyst regeneration process is therefore referred to as a hot regenerated zeolite-comprising catalyst. Preferably, the temperature of the zeolite-comprising catalyst retrieved in step (c) is lower than the temperature of the hot regenerated zeolite-comprising catalyst, preferably the temperature of the zeolite-comprising catalyst retrieved in step (c) is in the range of from 10 to 200° C., more preferably of from 50 to 95° C., below the temperature of the hot regenerated zeolite-comprising catalyst. This hot regenerated zeolite-comprising catalyst comprises less carbon deposits than the zeolite-comprising catalyst retrieved from the gas/solid separator(s), i.e. on a weight basis compared to the whole zeolite-comprising catalyst. It is not necessary to remove all the coke from the catalyst as it is believed that complete removal of the coke may lead to degradation of the zeolite.

An additional advantage of cooling the zeolite-comprising catalyst is that cooled zeolite-comprising catalyst is provided to the regenerator with a lower temperature upon entry into the regenerator, i.e. compared to zeolite-comprising catalyst, which was not cooled in the separator. As a result the temperature of the zeolite-comprising catalyst in the regenerator will also be lower. By exposing the zeolite-comprising catalyst to lower temperatures in the regenerator, thermally induced deactivation of the zeolite-comprising catalyst may be reduced.

A first part of this hot catalyst is subsequently provided to the first reactor to ensure a sufficient base temperature for the OTO process, where another part of the hot regenerated zeolite-comprising catalyst is provided to the second reactor. The hot regenerated zeolite-comprising catalyst is low in carbon, which is beneficial to the selectively of the OCP reaction, while the heat contained in the hot catalysts may be used to maintain, to at least a certain extent, the endothermic OCP process.

In the gas/solid separator, the gaseous product is separated from the zeolite-comprising catalyst. The gaseous product is preferably further treated to retrieve several product fractions from the gaseous product. The product fractions will preferably comprise one or more fractions comprising ethylene and/or propylene. The separation of the gaseous product in the mentioned fractions may be done using any suitable work-up section. The design of the work-up section depends on the exact composition of the olefinic product stream, and may include several separation steps. The design of such a work-up section is well known in the art and does not require further explanation.

Preferably, the product fractions will also comprise one or more fractions comprising C4+ olefins and in particular C4 and C5 olefins. These C4+ olefins and in particular C4 and C5 olefins may be provided to the OCP process as part of the olefin feed. In addition, external, i.e. not obtained from the gaseous product, olefins may be provided as part of the olefin feed.

Preferably, rather than sending the C4+ olefins in the gaseous product to the OCP process, the C4+ olefins are separated into at least a fraction comprising C4 olefins and a fraction comprising C5 olefins. The fraction comprising C4 olefins is recycled back to the first reactor together with or as part of the oxygenate feed to be contacted in the OTO process with the zeolite-comprising catalyst together with the oxygenate, while the fraction comprising C5 olefins is sent to the OCP.

Without wishing to be bound by any particular theory, it is believed that the cracking behaviour of C4 olefins and C5 olefins, when contacted with a zeolite-comprising catalyst, is different, in particular above 500° C. The cracking of C4 olefins is an indirect process which involves a primary oligomerisation process to a C8, C12 or higher olefin followed by cracking of the oligomers to lower molecular weight hydrocarbons including ethylene and propylene, but also, amongst other things, to C5 to C7 olefins, and by-products such as C2 to C6 paraffins, cyclic and aromatics. In addition, the cracking of C4 olefins is prone to coke formation, which places a restriction on the obtainable conversion of the C4 olefins. Generally, paraffins, cyclics and aromatics are not formed by cracking. They are formed by hydrogen transfer reactions and cyclisation reactions. This is more likely in larger molecules. Hence the C4 olefin cracking process, which as mentioned above includes intermediate oligomerisation, is more prone to by-product formation than direct cracking of C5 olefins. The conversion of the C4 olefins is typically a function of the temperature and space time (often expressed as the weight hourly space velocity, [kd_{C4-feed}/(kg_{catalyst}·hr)]).

With increasing temperature and decreasing weight hourly space velocity (WHSV) conversion of the C4 olefins in the feed to the OCP increases. Initially, the ethylene and propylene yields increase, but, at higher conversions, yield decreases at the cost of a higher by-product make and, in particular, a higher coke make, limiting significantly the maximum yield obtainable.

Contrary to C4 olefins, C5 olefin cracking is ideally a relatively straight forward-process whereby the C5 olefin cracks into a C2 and a C3 olefin, in particular above 500° C. This cracking reaction can be run at high conversions, up to 100%, while maintaining, at least compared to C4 olefins, high ethylene and propylene yields with a significantly lower by-product and coke make. Although, C5+ olefins can also oligomerise, this process competes with the more beneficial cracking to ethylene and propylene.

In a preferred embodiment of the process according to the present invention, instead of cracking the C4 olefins in the OCP reactor, the C4 olefins are recycled to the OTO reactor. Again without wishing to be bound by any particular theory, it is believed that in the OTO reactor the C4 olefins are alkylated with, for instance, methanol to C5 and/or C6 olefins. These C5 and/or C6 olefins may subsequently be converted into at least ethylene and/or propylene. The main by-products from this OTO reaction are again C4 and C5 olefins, which can be recycled to the OTO reactor and OCP reactor, respectively.

Therefore, preferably, where the gaseous products further include C4 olefins, at least part of the C4 olefins are provided to (i) the first reactor together with or as part of the oxygenate feed, and/or (ii) the second reactor as part of the olefin feed, more preferably at least part of the C4 olefins is provided to the first reactor together with or as part of the oxygenate feed.

Preferably, where the gaseous products further include C5 olefins, at least part of the C5 olefins are provided to the second reactor as part of the olefin feed. Preferably, the olefin feed to the second reactor comprises C4+ olefins, preferably C5+ olefins, more preferably C5 olefins.

Where a product fraction is recycled to either the first or second reactor, it is preferred to withdraw at least a part of the fraction to purge paraffins or other non or slowly reacting species that may be present in the fraction to prevent a buildup of these in the process.

Although less desired, the gaseous product will typically comprise some aromatic compounds such as benzene, toluene and xylenes. Although it is not the primary aim of the process, xylenes can be seen as a valuable product. Xylenes are amongst others formed in the OTO process by the alkylation of benzene and, in particular, toluene with oxygenates such as methanol. Therefore, in a preferred embodiment, a separate fraction comprising aromatics, in particular benzene, toluene and xylenes is separated from the gaseous product and at least in part recycled to the first reactor as part of the oxygenate feed. Preferably, part or all of the xylenes in the fraction comprising aromatics are withdrawn from the process as a product prior to recycling the fraction comprising aromatics to the first reactor.

The oxygenate feed provided to the OTO process in the first reactor, i.e. step (a), comprises oxygenate. The oxygenate used in the oxygenate feedstock provided to the OTO process is preferably an oxygenate which comprises at least one oxygen-bonded alkyl group. The alkyl group preferably is a C1-C5 alkyl group, more preferably C1-C4 alkyl group, i.e. comprises 1 to 5, or 1 to 4 carbon atoms respectively; more preferably the alkyl group comprises 1 or 2 carbon atoms and most preferably one carbon atom. Examples of oxygenates that can be used in the oxygenate feedstock include alcohols and ethers. Examples of preferred oxygenates include alcohols, such as methanol, ethanol, propanol; and dialkyl ethers, such as dimethylether, diethyl ether, methylethyl ether. Preferably, the oxygenate is methanol or dimethylether, or a mixture thereof.

Preferably the oxygenate feedstock comprises at least 50 wt. % of oxygenate, in particular methanol and/or dimethylether, based on total hydrocarbons, i.e. hydrocarbons including oxygenates, more preferably at least 70 wt. %.

Preferably, the oxygenate feed comprises oxygenate and olefins, more preferably oxygenate and olefins in an oxygenate:olefin molar ratio in the range of from 1000:1 to 1:1, preferably 100:1 to 1:1. More preferably, in a oxygenate:olefin molar ratio in the range of from 20:1 to 1:1, more preferably in the range of 18:1 to 1:1, still more preferably in the range of 15:1 to 1:1, even still more preferably in the range of 12:1 to 1:1. As mentioned above, it is preferred to convert a C4 olefin together with an oxygenate, to obtain a high yield of ethylene and propylene, therefore preferably at least one mole of oxygenate is provided for every mole of C4 olefin.

In the first reactor, the oxygenate feed is contacted with the zeolite-comprising catalyst. The oxygenate feed is contacted with the catalyst at a temperature in the range of from 350 to 700° C., preferably of from 450 to 650° C., more preferably of from 530 to 620° C., even more preferably of from 580 to 610° C.; and a pressure in the range of from 0.1 kPa (1 mbara) to 5 MPa (50 bara), preferably of from 100 kPa (1 bara) to 1.5 MPa (15 bara), more preferably of from 100 kPa (1 bara) to 300 kPa (3 bara). Reference herein to pressures is to absolute pressures.

In the second reactor, the olefin feed is contacted in the OCP process with the, hot, regenerated zeolite-comprising catalyst. The olefin feed provided to the OCP process in the second reactor, i.e. step (b), comprises olefin. The olefin used in the olefin feedstock provided to the OCP process is preferably an olefin obtained from the gaseous product. Preferably, the olefins include C4+ olefins, more preferably C5+ olefins, even more preferably C5 and C6 olefins, still more preferably include C5 olefins.

Preferably the olefin feedstock comprises at least 50 wt. % of olefin, in particular C5 olefin, based on total hydrocarbons, more preferably at least 70 wt. %.

The olefin feed is contacted with the catalyst at a temperature in the range of from 500 to 700° C., preferably of from 550 to 650° C., more preferably of from 550 to 620° C., even more preferably of from 580 to 610° C.; and a pressure in the range of from 0.1 kPa (1 mbara) to 5 MPa (50 bara), preferably of from 100 kPa (1 bara) to 1.5 MPa (15 bara), more preferably of from 100 kPa (1 bara) to 300 kPa (3 bara). Reference herein to pressures is to absolute pressures.

As mentioned above, preferably, both the first and second reactors are operated as riser reactors. The primary operators for controlling the reaction inside the reactor, and in particular a riser reactor, are the gas residence time, the cat/oil ratio and the feed and catalyst inlet temperature. The gas residence time and the cat/oil ratio may be correlated to the earlier mentioned WHSV.

The gas residence time herein refers to the average time it takes for gas at the reactor, inlet to reach the reactor outlet. The gas residence time is also referred to as τ.

The dimensionless cat/oil ratio herein refers to the mass flow rate of catalyst (kg/h) divided by the mass flow rate of the feed (kg/h), wherein the flow rate of the feed is calculated on a CH₂ basis.

Preferably, the first and second reactors are operated under similar temperature conditions. As the reactions taking place in the first reactor are primarily exothermic, whereas the reactions taking place in the second reactor are primarily endothermic, it is preferred that the feed and/or catalyst inlet temperature to the second reactor is higher than the temperature of the feed and/or catalyst inlet temperature to the first reactor. In order to maintain the temperature in the second reactor, heat must be provided to the second reactor. This may be done by providing the hot regenerated zeolite-comprising catalyst to the second reactor. Additionally, the feed to the second reactor may be provided at a higher temperature. The catalyst recirculation rate between the second reactor and the catalyst regenerator may be increased to provide more heat to the reactor.

In addition to the oxygenates and olefins, also an amount of diluent is provided to the first reactor and the second reactor together with or as part of the oxygenate feed and olefin feed, respectively.

During the conversion of the oxygenates in the first reactor, steam is produced as a by-product, which serves as an in-situ produced diluent. Typically, additional steam is added as diluent. The amount of additional diluent that needs to be added depends on the in-situ water make, which in turn depends on the composition of the oxygenate feed. Where the diluent provided to the first reactor is water or steam, the molar ratio of oxygenate to diluent is between 10:1 and 1:20. Other suitable diluents include inert gases such as nitrogen or methane, but may also include C2-C3 paraffins.

A diluent may also be provided to the second reactor together with the olefins. Preferably, the diluent provided to the second reactor is water or steam. Other suitable diluents include inert gases such as nitrogen or methane, but may also include C2-C3 paraffins. Preferably, the diluents provided to the first and second reactor are the same, more preferably water or steam.

The zeolite-comprising catalyst is a zeolite-comprising catalyst suitable for converting the oxygenates and olefins in respectively the first and second reactor and preferably includes zeolite-comprising catalyst compositions. Such zeolite-comprising catalyst compositions typically also include binder materials, matrix material and optionally fillers. Suitable matrix materials include clays, such as kaolin. Suitable binder materials include silica, alumina, silica-alumina, titania and zirconia, wherein silica is preferred due to its low acidity.

Zeolites preferably have a molecular framework of one, preferably two or more corner-sharing [TO₄] tetrahedral units, more preferably, two or more [SiO₄], [AlO₄] tetrahedral units.

The first and second zeolite-comprising catalysts suitable for converting the reactants in respectively the first and second reactors include those catalyst containing a zeolite of the ZSM group, in particular of the MFI type, such as ZSM-5, the MTT type, such as ZSM-23, the TON type, such as ZSM-22, the MEL type, such as ZSM-11, the FER type. Other suitable zeolites are for example zeolites of the STF-type, such as SSZ-35, the SFF type, such as SSZ-44 and the EU-2 type, such as ZSM-48.

The above mentioned zeolite-comprising catalysts are suitable for use in both the first and the second reactor. Under the appropriate reaction condition, these catalysts may induce the cracking of olefins as well as the conversion of oxygenates alone or together with C4 olefins to ethylene and propylene. These zeolite-comprising catalysts, in particular the ZSM zeolite-comprising catalyst have an advantage over for instance non-zeolite-comprising catalyst such as silicoaluminophosphates like SAPO-34. Although both types of catalyst are suitable to convert oxygenates to olefins, non-zeolite-comprising catalyst are less suitable for cracking olefins or converting oxygenates together with olefins such a C4 olefins. The advantage of using zeolites compared to e.g. silicoaluminophosphates becomes even more pronounced when the olefins include iso-olefins such as isobutene.

Preferred catalysts comprise a more-dimensional zeolite, in particular of the MFI type, more in particular ZSM-5, or of the MEL type, such as zeolite ZSM-11. The zeolite having more-dimensional channels has intersecting channels in at least two directions. So, for example, the channel structure is formed of substantially parallel channels in a first direction, and substantially parallel channels in a second direction, wherein channels in the first and second directions intersect. Intersections with a further channel type are also possible. Preferably the channels in at least one of the directions are 10-membered ring channels. A preferred MFI-type zeolite has a Silica-to-Alumina ratio SAR of at least 60, preferably at least 80.

The zeolite-comprising catalyst may comprise more than one zeolite. In that case it is preferred that the catalyst comprises at least a more-dimensional zeolite, in particular of the MFI type, more in particular ZSM-5, or of the MEL type, such as zeolite ZSM-11, and a one-dimensional zeolite having 10-membered ring channels, such as of the MTT and/or TON type.

The zeolite-comprising catalyst may comprise phosphorus as such or in a compound, i.e. phosphorus other than any phosphorus included in the framework of the zeolite. It is preferred that a catalyst comprising a MEL or MFI-type zeolite additionally comprises phosphorus. The phosphorus may be introduced by pre-treating the MEL or MFI-type zeolites prior to formulating the catalyst and/or by post-treating the formulated catalyst comprising the MEL or MFI-type zeolites. Preferably, the catalyst comprising MEL or MFI-type zeolites comprises phosphorus as such or in a compound in an elemental amount of from 0.05 to 10 wt % based on the weight of the formulated catalyst. A particularly preferred catalyst comprises phosphor and MEL or MFI-type zeolites having SAR of in the range of from 60 to 150, more preferably of from 80 to 100. An even more particularly preferred catalyst comprises phosphor and ZSM-5 having SAR of in the range of from 60 to 150, more preferably of from 80 to 100.

It is preferred that zeolites in the hydrogen form are used in the zeolite-comprising catalyst, e.g., HZSM-5, HZSM-11, and HZSM-22, HZSM-23. Preferably at least 50 wt %, more preferably at least 90 wt %, still more preferably at least 95 wt % and most preferably 100 wt % of the total amount of zeolite used is in the hydrogen form.

It is well known in the art how to produce such zeolites in the hydrogen form.

Preferably, the zeolite-comprising catalyst containing phosphorus has been prepared by a process which includes at least the following steps:

i) preparing an aqueous slurry comprising a zeolite, clay material and binder;
ii) spraydrying the aqueous slurry to obtain zeolite-comprising catalyst particles;
iii) treating the spraydried zeolite-comprising catalyst particles with phosphoric acid to introduce phosphorus compounds on the spraydried and zeolite-comprising catalyst particles; and
iv) calcining the spraydried zeolite- and phosphorus-comprising catalyst particles.

Preferably, the residence time of the reactants in the first reactor, also referred to as τ, is in the range of from 1 to 10 seconds, more preferably of from 3 to 6 seconds, even more preferably of from 3.5 to 4.5 seconds.

Preferably, the cat/oil ratio i.e. on a CH₂ basis for hydrocarbons including oxygenates, in the first reactor is in the range of from 1 to 100, more preferably of from of from 1 to 50, even more preferably 5 to 25.

It is preferable to control the severity of the process in the first reactor. When the process is operated at a too high severity, side reactions increase as well as by-product formation at the cost of ethylene and propylene selectivity. In case, the severity is too low, the process is operated inefficiently and sub optimal conversions are obtained. The severity of the process is influenced by several reaction and operation conditions; however a suitable measure for the severity of the process in the first reactor is the C5 olefin content in the first reactor effluent. A higher C5 olefin content indicates lower severity and vice versa. Preferably, the reaction conditions in the first reactor are chosen such that the first effluent stream comprises in the range of from 2.5 to 40 wt % of C5 olefins, based on the hydrocarbons in the reactor effluent, preferably 4 to 15 wt % of C5 olefins. The C5 content in the first reactor effluent depends on the severity of the reaction which may be controlled by changing one of more of the reaction conditions. One such condition is the temperature in the first reactor. As the temperature is reduced the C5 olefin content of the first reactor effluent may increase and vice versa where the aim is to reduce the C5 olefin content of the first reactor effluent. Furthermore, reducing the residence time of the reactants in the first reactor may also increase the C5 olefin content in the first reactor effluent and vice versa where the aim is to reduce the C5 olefin content of the first effluent. Alternatively, reducing the cat/oil ratio may also increase the C5 olefin content in the first reactor effluent and vice versa. One other way of increasing the C5 content in the first reactor effluent is by using a less active catalyst. This may be achieved by either operating the process with a catalyst having a higher average coke load or by reducing the catalyst refreshment rate, i.e. the rate of replacement of spent catalyst by fresh catalyst. Where the aim is to reduce the C5 olefin content of the first reactor effluent, the catalyst activity may be increased by the reverse of these measures. It will be appreciated that any combination of the above described measures may influence the C5 olefin content of the first effluent. It is well within the skills of the person skilled in the art to select the most appropriate measure. Preferably, the C5 olefin content of the first reactor effluent is controlled by adjusting the residence time and/or the cat/oil ratio, as these are adjusted most conveniently. As mentioned above, in case the C5 content in the first reactor effluent is higher than preferred, the above described measures may be used mutatis mutandis, i.e. increased temperature, residence time, cat/oil ratio and catalyst activity. Two or more of the above described measures may be used, in addition to others, to control the C5 content in the first reactor effluent. The C5 content in the first reactor effluent is conveniently analyzed using any suitable means of analyzing the hydrocarbon content in a process stream. Particular suitable means of analyzing the C5 content in the first reactor effluent include gas chromatography and near infrared spectrometry.

Preferably, the reaction conditions in the first reactor are chosen such that the oxygenate conversion is in the range of from 90 to 100%, based on the oxygenates provided to the first reactor, preferably 95 to 100%.

Preferably, the residence time of the reactants in the second reactor, also referred to as τ, is in the range of from 1 to 10 seconds, more preferably of from 3 to 6 seconds, even more preferably of from 3.5 to 4.5 seconds.

Preferably, the cat/oil ratio in the second reactor is in the range of from 1 to 100, more preferably of from 1 to 50, even more preferably of from 5 to 25.

Typically, the catalyst deactivates in the course of the process, amongst other things due to deposition of coke on the catalyst.

The catalyst particles used in the process of the present invention can have any shape known to the skilled person to be suitable for this purpose, for it can be present in the form of spray dried catalyst particles, spheres, tablets, rings, extrudates, etc. Extruded catalysts can be applied in various shapes, such as, cylinders and trilobes. Spray-dried particles allowing use in a fluidized bed or riser reactor system are preferred. Spherical particles are normally obtained by spray drying. Preferably the average particle size is in the range of 1-200 μm, preferably 50-100 μm. Typically and preferably, Geldart A-class particles are used where the reactors are riser reactors, see D. Kunii and O. Levenspiel, Fluidization Engineering, 2^{nd} Ed, Butterworth-Heineman, Boston, London, Singapore, Sydney, Toronto, Wellington, 1991, p 77 for Geldart classification of particles.

The invention also provides reaction system suitable for preparing ethylene and propylene. The system according to the invention is herein below explained in more detail with reference to the non-limiting FIG. 1.

The system (10) according to the invention comprises a first reactor (20), a second reactor (30), gas/solid separator (40) and a regenerator (50). The first reactor (20) comprises one or more inlets (22) for receiving oxygenate feed and diluent, an inlet (24) for receiving hot regenerated zeolite-comprising catalyst from regenerator (50), an inlet (26) for receiving zeolite-comprising catalyst from gas/solid separator (40) and an outlet (28) for retrieving a first reactor effluent.

The second reactor (30) comprises one or more inlets (32) for receiving olefin feed and diluent, an inlet (34) for receiving hot regenerated zeolite-comprising catalyst from regenerator (50) and an outlet (36) for retrieving a second reactor effluent.

Preferably, at least the first reactor comprises one or more riser reactors operated in parallel. Optionally, also the second reactor comprises one or more riser reactors operated in parallel. This allows for the increase of the capacity of the reactors without the need to construct riser reactors with very large diameters to attain the desired capacity.

The gas/solid separator (40) comprises an inlet (42) for receiving the first reactor effluent stream and an inlet (44) for receiving the second reactor effluent stream. Optionally, the first reactor effluent stream and the second reactor effluent stream may be provided to the gas/solid separator (40) through the same inlet. Gas/solid separator (40) further comprises an outlet (46a) for zeolite-comprising catalyst, an outlet (46b) for zeolite-comprising catalyst and an outlet (48) for the gaseous product.

Preferably, the gas/solid separator (40) comprises cooling means, such as a water quench.

Preferably, the gas/solid separator (40) comprises a primary cyclone, a secondary cyclone and a stripper. Where the primary and secondary cyclones serve to remove zeolite-comprising catalyst from the gaseous product, the later stripper uses a stripping medium such as steam to remove residual gaseous product from the zeolite-comprising catalyst.

The regenerator (50) comprises vessel (52) and a stripper section (54) in fluid communication with vessel (52). Zeolite-comprising catalyst from gas/solid separator (40) may enter vessel (52) via inlet (56). Oxygen, for instance in the form of air, is provided via inlet (58). Flue gas may exit vessel (52) via outlet (60). The zeolite-comprising catalyst may pass from vessel (52) to stripper section (54), where it may be stripped with a stripping medium such as nitrogen, provided via inlet (62), to remove residual oxygen. The zeolite-comprising catalyst is retrieved from the stripper section (54) of regenerator (50) via outlets (64) and (66).

In order to provide the desired catalyst circulation as described in the process according to the invention, the system according to the invention further comprises: means (70) for providing a first reactor effluent stream from the first reactor (20) to the gas/solid separator (40); means (75) for providing a second reactor effluent stream from the second reactor (30) to the gas/solid separator (40); means (80) for providing zeolite-comprising catalyst from the gas/solid separator (40) to the regenerator (50); means (85) for providing zeolite-comprising catalyst from the regenerator (50) to the first reactor (20); means (90) for providing zeolite-comprising catalyst from the regenerator (50) to the second reactor (30); and means (95) for providing zeolite-comprising catalyst from the gas/solid separator (40) to the first reactor (20).

The means (70, 75, 80, 85, 90, 95) may be any suitable means for providing the mentioned solids, gases or liquids from one unit in the system to the other. Typically these means are conduit, pipes or the like.

As can be seen from FIG. 1, preferably, means (70) fluidly connects outlet (28) with inlet (42), means (75) fluidly connects outlet (36) with inlet (44); means (80) fluidly connects inlet (56) with outlet (46b); means (85) fluidly connects inlet (24) with outlet (64); means (90) fluidly connects inlet (34) with outlet (66) and means (95) fluidly connects inlet (26) with outlet (46a).

Typically, system (10) further comprises means (100) to provide oxygenate feed to first reactor (20), via inlet (22), and means (105) to provide olefin feed to second reactor (30), via inlet (32). The gaseous product retrieved from gas/solid separator (4) via outlet (48) may be provided to a separation section (106) via means (110). In separation section (106) the gaseous product is treated to remove steam and water and to separate the remainder into the desired product fractions. Such treatment may include for instance a water quench to remove steam and one or more compression steps to compress the gaseous product. Typically, at least one or more fractions comprising ethylene and propylene are retrieved from separation section 106 via means 112. However, preferably, also a fraction comprising C4 olefins is retrieved via means (114) and provided to means (100) to form part of the oxygenate feed. In addition, preferably, also a fraction comprising C5 olefins is retrieved via means (116) and provided to means (105) to form part of the olefin feed.

The invention further provides the use of the reaction system according to the invention in a process according to the invention.

1. A process for preparing ethylene and/or propylene, wherein oxygenates and olefins are converted to ethylene and/or propylene over a zeolite-comprising catalyst, comprising the steps of:
a) reacting in a first reactor an oxygenate feed over the zeolite-comprising catalyst at a temperature in the range of from 350 to 1000° C. and retrieving from the first reactor a first reactor effluent stream comprising gaseous products, including ethylene and/or propylene, and zeolite-comprising catalyst;
b) reacting in a second reactor an olefin feed over the zeolite-comprising catalyst at a temperature in the range of from 500 to 700° C. and retrieving from the second reactor a second reactor effluent stream comprising gaseous products, including ethylene and/or propylene, and zeolite-comprising catalyst;
c) providing the first and second reactor effluent stream to one or more gas/solid separators to retrieve zeolite-comprising catalyst from the first and second reactor effluent;
d) providing part of the zeolite-comprising catalyst retrieved in step (c) to the first reactor;
e) regenerating another part of the zeolite-comprising catalyst retrieved in step (c) by contacting the zeolite-comprising catalyst with oxygen at elevated temperatures to provide a hot regenerated zeolite-comprising catalyst; and
f) providing part of the hot regenerated zeolite-comprising catalyst to the first reactor and another part of the hot regenerated zeolite-comprising catalyst to the second reactor.

2. A process according to claim 1, wherein at least part of the first and second effluent are provided to the same gas/solid separator.

3. A process according to claim 1, wherein the first reactor and/or the second reactor is a riser reactor.

4. A process according to claim 1, wherein the zeolite-comprising catalyst is cooled in the gas/solid separator.

5. A process according to claim 1, wherein the temperature of the zeolite-comprising catalyst retrieved in step (c) is lower than the temperature of the hot regenerated zeolite-comprising catalyst.

6. A process according to claim 1, wherein the gaseous products further include C4 olefins and at least part of the C4 olefins are provided to (i) the first reactor together with or as part of the oxygenate feed, and/or (ii) the second reactor as part of the olefin feed.

7. A process according to claim 1, wherein the gaseous products further include C5 olefins and at least part of the C5 olefins are provided to the second reactor as part of the olefin feed.

8. A process according to claim 1, wherein the oxygenate feed comprises methanol and/or dimethylether.

9. A process according to claim 1, wherein the olefin feed comprises C4+ olefins.

10. A process according to claim 1, wherein the zeolite-comprising catalyst comprises ZSM-5.

11. A reaction system suitable for preparing ethylene and propylene, comprising
a) a first reactor;
b) a second reactor;
c) a regenerator; and
d) a gas/solid separator;
wherein the reaction system further comprises:
means for providing a first reactor effluent stream from the first reactor to the gas/solid separator;
means for providing a second reactor effluent stream from the second reactor to the gas/solid separator;
means for providing zeolite-comprising catalyst from the gas/solid separator to the regenerator;
means for providing zeolite-comprising catalyst from the regenerator to the first reactor;
means for providing zeolite-comprising catalyst from the regenerator to the second reactor; and
means for providing catalyst from the gas/solid separator to the first reactor.

12. A reaction system according to claim 11, wherein the first reactor comprises one or more riser reactors.

13. A reaction system according to claim 11, wherein the gas/solid separator comprises cooling means.

14. A reaction system according to any one of claims 11, wherein the gas/solid separator comprises a primary cyclone, a secondary cyclone and a stripper.

15. The use of the reaction system according to claim 11 in a process according to any one of claims 1 to 10
